(19) United States
(12) Patent Application Publication
(10) Pub. No.: US 2010/0086990 A1
(43) Pub. Date: Apr. 8, 2010

(54) APPARATUS AND METHOD FOR NUCLEIC ACID AMPLIFICATION

(75) Inventors: Keith Stanley, Darlinghurst (AU); John Corbett, New South Wales (AU)

Correspondence Address: Baker Donelson Bearman, Caldwell & Berkowitz, PC, 555 Eleventh Street, NW, Sixth Floor, Washington, DC, 20004, US

(73) Assignee: CORBETT RESEARCH PTY LTD, Mortlake (AU)

(21) Appl. No.: 12/529,551

(22) PCT Filed: Feb. 29, 2008
(86) PCT No.: PCT/AU2008/000274

§ 371 Date: Sep. 2, 2009

(30) Foreign Application Priority Data
Mar. 2, 2007 (AU) 2007901073

(51) Int. Cl. C12M 1/38; C12M 1/00

(52) U.S. Cl. 435/286.1; 435/294.1

(57) ABSTRACT

The present invention provides apparatus (1) for conducting sequential nucleic acid amplification reactions. The apparatus (1) comprises a platform (2) having a sample compartment (5) and a plurality of reaction compartments (10). The sample compartment (5) is adapted to receive a fluid sample for conducting a first amplification reaction and the platform (2) is adapted to substantially evenly distribute the fluid sample into the plurality of reaction compartments (10) for conducting second amplification reactions. The present invention also extends to a kit, a method and a device for conducting sequential nucleic acid amplification reactions.

### FIELD OF THE INVENTION

The present invention relates to a method and apparatus for nucleic acid amplification, and in particular for conducting sequential nucleic acid amplifications. However, it will be appreciated that the invention is not limited to this particular field of use.

### BACKGROUND OF THE INVENTION

The following discussion of the prior art is provided to place the invention in an appropriate technical context and enable the advantages of it to be more fully understood. It should be appreciated, however, that any discussion of the prior art throughout the specification should not be considered as an express or implied admission that such prior art is widely known or forms part of common general knowledge in the field.

PCR is a technique involving multiple cycles that results in the exponential amplification of certain polynucleotide sequences each time a cycle is completed. The technique of PCR is well known and is described in many books, including, PCR: A Practical Approach M. J. McPherson, et al., IRL Press (1991), PCR Protocols: A Guide to Methods and Applications by Innis, et al., Academic Press (1990), and PCR Technology: Principals and Applications for DNA Amplification H. A. Erlich, Stockton Press (1989). PCR is also described in many US patents, including U.S. Pat. Nos. 4,683,195; 4,683,202; 4,800,159; 4,965,188; 4,889,818; 5,075,216; 5,079,352; 5,104,792; 5,023,171; 5,091,310; and 5,066,584.

The PCR technique typically involves the step of denaturing a polynucleotide, followed by the step of annealing at least a pair of primer oligonucleotides to the denatured polynucleotide, i.e., hybridizing the primer to the denatured polynucleotide template. After the annealing step, an enzyme with polymerase activity catalyzes synthesis of a new polynucleotide strand that incorporates the primer oligonucleotide and uses the original denatured polynucleotide as a synthesis template. This series of steps (denaturation, primer annealing, and primer extension) constitutes a PCR cycle.

As cycles are repeated, the amount of newly synthesized polynucleotide increases exponentially because the newly synthesized polynucleotides from an earlier cycle can serve as templates for synthesis in subsequent cycles. Primer oligonucleotides are typically selected in pairs that can anneal to opposite strands of a given double-stranded polynucleotide sequence so that the region between the two annealing sites is amplified.

Denaturation of DNA typically takes place at around 90 to 95° C., annealing a primer to the denatured DNA is typically performed at around 40 to 60° C., and the step of extending the annealed primers with a polymerase is typically performed at around 70 to 75° C. Therefore, during a PCR cycle the temperature of the reaction mixture must be varied, and varied many times during a multicycle PCR experiment.

The PCR technique has a wide variety of biological applications, including for example, DNA sequence analysis, probe generation, cloning of nucleic acid sequences, site-directed mutagenesis, detection of genetic mutations, diagnoses of viral infections, molecular “fingerprinting” and the monitoring of contaminating microorganisms in biological fluids and other sources.

In addition to PCR, other in vitro amplification procedures, including ligase chain reaction as disclosed in U.S. Pat. No. 4,988,617 to Landegren and Hood, are known and advantageously used in the prior art. More generally, several important methods known in the biotechnology arts, such as nucleic acid hybridization and sequencing, are dependent upon changing the temperature of solutions containing sample molecules in a controlled fashion. Conventional techniques rely on use of individual wells or tubes cycled through different temperature zones. However, continuous flow methods have also been proposed. These devices, however, require the use of high-pressure pumps for moving fluid through a narrow-bore tube, which limits the usefulness of these devices for miniaturizing and automating the PCR. Nevertheless automation and miniaturization of the performance of these methods remain desirable goals in the art, particularly under conditions where a large multiplicity of samples must be analyzed simultaneously or when there is a small amount of sample to be analyzed. Further, it would be desirable to provide devices and methods that provide reduced sample handling and thereby reduce the possibility of contamination, particularly of DNA samples, while being able to use relatively inexpensive laboratory equipment.

It is an object of the present invention to overcome or ameliorate at least one of the disadvantages of the abovementioned prior art, or to provide a useful alternative.

### SUMMARY OF THE INVENTION

According to a first aspect the present invention provides apparatus for conducting sequential nucleic acid amplification reactions, the apparatus comprising:

a platform comprising:
a sample compartment adapted to receive a fluid sample for conducting a
first amplification reaction, and
a plurality of reaction compartments,

said platform being adapted to substantially evenly distribute said fluid sample into said plurality of reaction compartments for conducting second amplification reactions.

According to a second aspect the present invention provides a method for conducting sequential nucleic acid amplification reactions, comprising:

providing a platform comprising:
a sample compartment, and
a plurality of reaction compartments, charging said sample compartment with a fluid sample and conducting a first amplification reaction, and

substantially evenly distributing said fluid sample into said plurality of reaction compartments for conducting second amplification reactions.

According to a third aspect the present invention provides a method for conducting sequential nucleic acid amplification reactions, comprising: providing apparatus according to the first aspect, charging said sample compartment with a fluid sample and conducting a first amplification reaction, and substantially evenly distributing said fluid sample into said plurality of reaction compartments for conducting second amplification reactions.

In one example, the first amplification reaction uses a multiplicity of primers (multiplexed primer pairs) to simultaneously amplify a number of targets up to a point where competition has not occurred. The product from this first amplification reaction is then optionally diluted (discussed further below) and distributed to the reaction compartments for the second amplification reaction. Each of the reaction compartments preferably contains a single pair of primers. As will be appreciated, the apparatus and method according to the present invention provides the considerable advantage of reducing manual intervention thereby reducing the possibility of contamination when optionally diluting and distributing the product of the first amplification reaction. The person skilled in the art will appreciate the many other advantages which follow from use of the apparatus and method of the invention.

Preferably the platform is rotatable and the fluid sample is substantially evenly distributable from the sample compartment into the plurality of reaction compartments upon the application of centrifugal force. However, it will be appreciated that fluid sample may be substantially evenly distributed from the sample compartment into the plurality of reaction compartments by other means. Preferably the plurality of reaction compartments are positioned radially outwards of the sample compartment. Preferably the plurality of reaction compartments comprises 2 or more reaction compartments which are distributed at the periphery of the rotatable platform in an array.

In one embodiment, the fluid sample is substantially evenly distributed to the plurality of radially outer reaction compartments through, or by way of, a metering manifold, which is positioned fluidly intermediate the reaction compartments and the sample compartment. Preferably the sample compartment is in fluid communication with the metering manifold by a channel, and the metering manifold is, in turn, in fluid communication with the plurality of radially outer reaction compartments by a plurality of respective channels. It will be appreciated that the terms “fluid communication” and “fluidly connected” are interchangeable, and should be construed as a passageway between the compartments (or manifold).

It will be appreciated that a first amplification reaction can be conducted in the radially inner sample compartment, and when distributed or metered to the plurality of radially outer reaction compartments, a second amplification reaction can then be conducted. It will further be appreciated that the fluid sample which is the result of the second amplification reaction could also be optionally diluted and metered to an array of secondary reaction compartments for conducting third amplification reactions, or undergo a further chemical reaction.

In another embodiment, each of the reaction compartments are fluidly connected to the metering manifold by a channel having a selective flow means in the form of a valve for selectively permitting a fluid flow therethrough. In further embodiments the sample compartment is also fluidly connected to the metering manifold by a channel having a selective flow means for selectively permitting a fluid flow therethrough.

In one embodiment, the metering manifold may simultaneously act as the dilution compartment for diluting the reacted fluid sample. In this embodiment the metering manifold may contain a dilution fluid, and the fluid sample introduced to the metering manifold is diluted and then substantially evenly metered to the plurality of radially outer reaction compartments. However, in an alternative embodiment a separate dilution compartment is provided fluidly intermediate the sample compartment and the metering manifold. In this embodiment the radially inner sample compartment is in fluid communication with a dilution compartment, which in turn is in fluid communication with the metering manifold, which in turn is in fluid communication with the array of radially outer reaction compartments.

In a further embodiment, the present invention provides apparatus for conducting sequential nucleic acid amplifications, comprising: a rotatable platform having a plurality of radially inner sample compartments for each receiving one of a plurality of fluid samples and conducting first amplification reactions, each said sample compartment being in fluid communication with a respective dilution compartment, each said dilution compartment being in fluid communication with a respective metering manifold for substantially evenly distributing each said diluted fluid sample into a respective array of radially outer reaction compartments for conducting a plurality of second amplification reactions, each said reaction compartment being fluidly connected to a respective metering manifold by a channel having a selective flow means for selectively permitting a fluid flow therethrough, wherein the application of sufficient centrifugal force selectively transports said fluid sequentially from one compartment to the next.

The present invention also extends to a method for conducting sequential nucleic acid amplifications, comprising the steps of: providing apparatus according to the previous embodiment, charging said sample compartment with a fluid sample, conducting a first amplification reaction, charging said dilution compartment with a dilution liquid, applying sufficient centrifugal force to transport said fluid sample to said dilution compartment for diluting said sample, applying sufficient centrifugal force to transport said diluted fluid sample to said metering manifold, actuating said selective flow means, applying sufficient centrifugal force to substantially evenly distribute said diluted fluid sample to said reaction compartments, and conducting second amplification reactions.

In preferred embodiments the rotatable platform is a generally planar circular or annular disc. However, in alternative embodiments, the rotatable platform is a sector or segment of a circular/annular disc (i.e. a wedge) preferably having only 1 or 2 sample compartments (together with their respective sample/dilution/metering compartments) (discussed further below). It will be appreciated that the annular platform or the wedge-shaped platform as discussed above may be received in a complementary rotatable base which provides a centrifugal force for transporting fluid and optionally for heating/cooling the various compartments for conducting the nucleic acid amplifications. Alternatively, the compartments and channels are provided separately in kit form and they may be releasably captively retained in a rotatable base by complementary holding receptacles.

In one embodiment, substantially radially extending channels are provided for fluidly connecting the sample compartment to the dilution compartment, the dilution compartment to the metering manifold, and the metering manifold to the plurality of reaction compartments. Channels in series fluidly connect the sample compartment, dilution compartment and metering manifold, whilst channels in parallel fluidly connect each of the reaction compartments with the metering manifold. However it will be appreciated that other configurations for fluidly connecting the compartments will be possible to similar effect. In an alternative embodiment, the plurality of reaction compartments are fluidly connected in series, i.e. a first reaction compartment of said plurality of reaction compartments is fluidly connected to a second reaction compartment of said plurality of reaction compartments, which in turn is fluidly connected to a third reaction compartment of said plurality of reaction compartments, etc. In this embodiment, there is no need for a metering manifold and the first of the plurality of reaction compartments is fluidly connected to the dilution compartment. In an alternative yet somewhat similar embodiment, the plurality of reaction compartments are fluidly connected in parallel to a primary channel which is fluidly connected to the dilution compartment. In these last two embodiments, the diluted fluid sample is transported to the last of the plurality of reaction compartments under centrifugation and then “back-fills” the preceding reaction compartments.

In preferred embodiments the platform is a circular or annular disc wherein the sample compartment, dilution compartment, metering manifold, reaction compartments and channels are embedded in the surface of the disc, for example as shown in the Figures. However, it will be appreciated that other configurations are possible. For example, the compartments, manifold and channels may be relatively planar and embedded within the disc, such that the compartments, manifold and channels do not substantially protrude from the upper or lower surfaces of the disc.

The sample compartment is preferably disposed at a radially inner position and the plurality of reaction compartments are preferably substantially evenly circumferentially spaced about the periphery of the disc; the dilution compartment and metering manifold being disposed therebetween. This arrangement of compartments generally defines a sector of the disc, which may comprise a plurality of sectors. For example, the disc may include between about 2 to 20 sectors having about 72 reaction compartments in total. However, the disc may include as few as 18 and as many as 144 reaction compartments. In one example, 24 gene measurements may be made for a disc having 3 sectors and 72 reaction compartments. In another example, 12 gene measurements may be made for a disc having 6 sectors and 72 reaction compartments. However, as the skilled person will appreciate, the number of compartments may be tailored to suit the particular application/assay. Furthermore, the skilled person will appreciate that the number of sectors is proportional to the physical dimensions of the disc. Thus, the number of sectors is a function of the relative size of the various compartments and the physical dimensions of the disc.

As discussed above, in alternative embodiments the platform comprises only a sector/segment of a circular disc (i.e. a wedge) having the sample compartment, dilution compartment, metering manifold and reaction compartments embedded therein. This modular arrangement is useful for applications which only require the amplification of a small number of samples, e.g. 1 or 2, thereby avoiding the use of an entire disc having many sectors. However, in further embodiments the sample compartment, dilution compartment, metering manifold, reaction compartments and interconnecting channels are provided separately such that they can be fitted or clipped into receptacles in suitable rotor equipment (discussed further below).

The volumetric capacity of the sample compartment is from about 5 μL to about 20 μL and the dilution compartment from about 100 μL to about 500 μL. The total volume of the metering manifold is preferably about 1200 μL and the combined volume of the axially extending metering “funnels” disposed on the radially outer wall of the metering manifold totals about 100 μL to about 600 μL. Each of the reaction compartments has a volumetric capacity of about 20 μL to about 250 μL. It will be appreciated that the total combined volume of the reaction compartments is greater than the volume of the sample compartment plus the volume of the dilution compartment. Further, it will be appreciated that the combined volume of the axially extending metering “funnels” disposed on the radially outer wall of the metering manifold is greater than the total volume of the preceding reaction compartments, thereby ensuring that the fluid sample is substantially evenly distributed to the reaction compartments.

In preferred embodiments, the sample compartment and the reaction compartments are formed as substantially axially extending substantially cylindrically shaped wells. However, the dilution compartment and the metering manifold are preferably formed as substantially arcuate troughs (when viewed axially). Additionally, since during centrifugation under acceleration the fluid sample will tend to migrate to one end of the dilution compartment/trough (depending on the direction or rotation) the channel fluidly connecting the dilution compartment and the metering manifold is preferably disposed at a common end of these compartments, rather than centrally disposed. This configuration advantageously allows a selective flow between these compartments depending on the direction of rotation. Further, the sample compartment and/or metering manifold may have inclined wall portions such that the fluid tends to drain to the channel leading to the subsequent compartment when centrifugal force is applied.

A selective flow means is provided for selectively permitting a fluid flow from the metering manifold to the plurality of reaction compartments. The selective flow means is a preferably a wax valve having a predetermined melting temperature of about 95° C. Thus, raising the local temperature in the region surrounding the wax valve above the predetermined melting temperature melts the wax and opens the channel thereby to permit a fluid flow therethrough. In other embodiments the sacrificial valve may also be incorporated into the other channels (connecting the sample/dilution compartments, and the dilution compartment/metering manifold). Advantageously, melting the wax provides a surface seal on the fluid sample since the wax is of typically lower density than the fluid sample.

In embodiments where a wax valve is employed, the integrity of the wax valve may be affected if the entire apparatus is heated to effect the PCR reaction. Therefore, localised delivery of heat to the sample compartments may be required, for example by use of microwaves or infrared heating, such as disclosed in International PCT Publication No.'s WO 2003/093407 and WO 2003/102226.

As described above, the initial fluid sample is successively transported from the sample compartment to the dilution compartment, then to the metering manifold and finally to the reaction compartments. The preferred driving force for transporting the fluid from compartment to compartment is the application of centrifugal force by rotation of the platform, for example by Rotor-Gene® equipment marketed by Corbett Research (See International Patent Application No. PCT/AU98/00277). The platform may be rotated at a “low” speed to initially move the fluid sample from the sample compartment to the dilution compartment, and then at a “high” speed to move the diluted fluid sample from the dilution compartment to the metering manifold. The “low” speed may be about 200 to about 750 rpm and the “high” speed greater than about 800 rpm. However the skilled person will appreciate that other rotational speeds may be employed for similar effect. For example, the low speed may be 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, or 750 rpm, or in the range of about 200 to about 220, 220 to about 240, 240 to about 260, 260 to about 280, 280 to about 300, 300 to about 320, 320 to about 340, 340 to about 360, 360 to about 380, 380 to about 400, 400 to about 420, 420 to about 440, 440 to about 460, 460 to about 480, 480 to about 500, 500 to about 520, 520 to about 540, 540 to about 560, 560 to about 580, 580 to about 600, 600 to about 620, 620 to about 640, 640 to about 660, 660 to about 680, 680 to about 700, 700 to about 720, 720 to about 740, or about 740 to about 750 rpm. The high speed may be 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, or 1200 rpm, or in the range of about 800 to about 820, 820 to about 840, 840 to about 860, 860 to about 880, 880 to about 900, 900 to about 920, 920 to about 940, 940 to about 960, 960 to about 980, 980 to about 1000, 1000 to about 1020, 1020 to about 1040, 1040 to about 1060, 1060 to about 1080, 1080 to about 1100, 1100 to about 1120, 1120 to about 1140, 1140 to about 1160, 1160 to about 1180, or 1180 to about 1200 rpm.

The rotatable platform is preferably formed from a thermostable thermoplastics material such as polypropylene or polyethylene, which is preferably injection moulded. However, it will be appreciated that the apparatus of the invention could be machined out of, say, glass, or other suitable materials known in the art. Advantageously, the thermoplastics material may be transparent at predetermined wavelengths for allowing a light source/detector arrangement to monitor a reaction occurring in a compartment. Furthermore, the compartments, manifold and channels may include a raised upper edge for sealing engagement with a cover sheet, which is preferably a transparent thermally fusible plastic sheet having a pair of apertures such that when the sheet is fused to the upper edges the apertures provide access to the sample compartment for charging the compartment with a sample and the dilution compartment for charging the compartment with a dilution fluid. The reaction compartments may be pre-charged with amplification reagents and sealed with the cover sheet (since the channels leading into the reaction compartments are closed by virtue of the selective flow means).

Preferably the apertures have dimensions adapted to automated loading devices such as micropipettors, for example, a standard 200 μL plastic pipette tip having a tip diameter of 1.5 mm; micropipette tips of diameter 1 mm; piezoelectric or ceramic drop delivery systems; and inkjet-based fluid delivery systems.

In alternative configurations, the dilution compartment and metering manifold may be combined into a single dilution/metering manifold. To explain, a fluid sample is initially charged into a sample compartment and a first amplification reaction is conducted. A dilution fluid is charged into the dilution/metering manifold and sufficient centrifugal force is applied to transport the fluid sample to the dilution/metering manifold for dilution. The selective flow means may then be actuated and the diluted fluid sample transported via centrifugal force to the plurality of reaction compartments for conducting a plurality of second amplification reactions. As the skilled person will appreciate, a reciprocating “agitation” action may be applied to the apparatus for mixing the fluids in the dilution/metering manifold. Alternatively, the apparatus may be vibrated to effect mixing.

The sample and reaction compartments are adapted for thermal communication with a heat exchanger for conducting the first and second amplification reactions respectively. In one embodiment, pre-existing Rotor-Gene® thermal cycling equipment may be modified to accept the apparatus of the present invention. In this embodiment the rotation required to transport the fluid from compartment to compartment is computer controlled. For example, according to a fourth aspect the present invention provides a device for conducting nucleic acid amplification, comprising: a base adapted to receive the apparatus according to the first aspect, a rotating means for rotating said apparatus according to a pre-determined sequence to selectively transport fluid from one compartment to the next, temperature control elements in thermal communication with said sample and reaction compartments for providing a thermal profile sequence for conducting said first and second amplification reactions respectively, and a programmable controller for storing and controlling said sequences.

It will be appreciated that other equipment may be adapted for the present invention. For example, commercially available or pre-existing equipment may be adapted to receive the apparatus as described herein. This equipment may provide rotation of the apparatus, and suitable heat exchangers for conducting the nucleic acid amplifications may be retrofitted. Alternatively, the equipment may already be adapted for conducting the nucleic acid amplifications and may be retrofitted to provide rotation of the apparatus as described herein.

For example, whilst the RotorGene® equipment includes a rotatable base adapted for thermal cycling of a sample for nucleic acid amplification, other means of thermal cycling of a sample are anticipated. For example, infrared heaters may be used in conjunction with, say, the temperature sensing means as disclosed in International Patent Application No. PCT/AU03/00673 (Publication No. WO 2003/102522).

According to a fifth aspect the present invention provides a kit for conducting sequential nucleic acid amplifications, comprising: apparatus according to the first aspect; and reagents necessary for conducting the second amplification reactions contained in the reaction compartments. Alternatively, or additionally, in embodiments of the apparatus of the invention having a dilution compartment the dilution compartment contains reagents necessary for diluting the product of the first amplification reaction.

According to a sixth aspect, preferably the first amplification reaction comprises a multiplex PCR reaction and the second amplification reaction comprises a nested or hemi-nested PCR reaction.

In other embodiments, the platform comprises “home-flag”, that may be a reflective or absorbing stripe that can be positioned on the surface of the platform and sensed by an emitter/photodiode pair as the platform is spun, thus permitting the orientation of the platform with respect to the instrument to be determined Alternatively the home-flag is a tag.

In one embodiment, the plurality of reaction compartments are connected in parallel from the metering manifold, however in other embodiments the plurality of reaction compartments are connected in series from the metering manifold.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”.

### DEFINITIONS

In describing and claiming the present invention, the following terminology will be used in accordance with the definitions set out below. It is also to be understood that the terminology used herein is for the purpose of describing particular embodiments of the invention only and is not intended to be limiting. Unless defined otherwise, all technical and scientific terms used herein have the same meaning as commonly understood by one having ordinary skill in the art to which the invention pertains.

For the purposes of this invention, the term “sample” will be understood to encompass any fluid, solution or mixture, either isolated or detected as a constituent of a more complex mixture, or synthesized from precursor species. In particular, the term “fluid sample” will be understood to encompass any biological species of interest. The term “biological sample” or “biological fluid sample” will be understood to mean any biologically-derived sample, including but not limited to blood, plasma, serum, lymph, saliva, tears, cerebrospinal fluid, urine, sweat, plant and vegetable extracts, semen, and ascites fluid. It will be appreciated that the terms “fluid sample” and “reaction mixture” are synonymous.

For the purposes of this invention, the term “selective flow means” will be understood to mean a valve preferably made of a fungible material that can be selectively removed from the fluid flow path. In preferred embodiments, the selective flow means is a wax valve which is removed from the fluid flow path by heating, using any of a variety of heating means including infrared illumination and most preferably by activation of heating elements. One typical wax suitable for the present invention is paraffin wax. Preferred waxes are devoid of biological contaminants and have densities less than the sample.

For the purposes of this invention, the terms “in fluid communication” or “fluidly connected” are used interchangeably and are intended to define compartments/manifolds that are operably interconnected to allow fluid flow therebetween.

For the purposes herein, a “centrifugal force” is the apparent force drawing a rotating body away from the centre of rotation. The centrifugal force is equal and opposite to the centripetal force.

The term “channels in series” is intended to refer to an ordered linear arrangement of channels, and the term “channels in parallel” is intended to refer to an array of channels which are in a plurality of linear arrangements.

### BRIEF DESCRIPTION OF THE DRAWINGS

A preferred embodiment of the invention will now be described, by way of example only, with reference to the accompanying drawings in which:

FIG. 1a is a plan view of apparatus according to one embodiment of the invention in which the rotatable platform is an annular disc having 8 sample compartments (and each compartment's respective sample/dilution/metering compartments);

FIG. 1b is a plan view of apparatus according to another embodiment of the invention in which the rotatable platform is only a segment of an annular disc having only a single reaction compartment (together with its respective sample/dilution/metering compartments);

FIG. 2 is an under-side view of the apparatus as shown in FIG. 1;

FIG. 3 is an enlarged plan view of one sector of the apparatus as shown in FIG. 1;

FIG. 4 is a perspective view of the enlarged sector as shown in FIG. 3; and

FIG. 5 is a perspective under-side view of the device as shown in FIG. 4.

### PREFERRED EMBODIMENT OF THE INVENTION

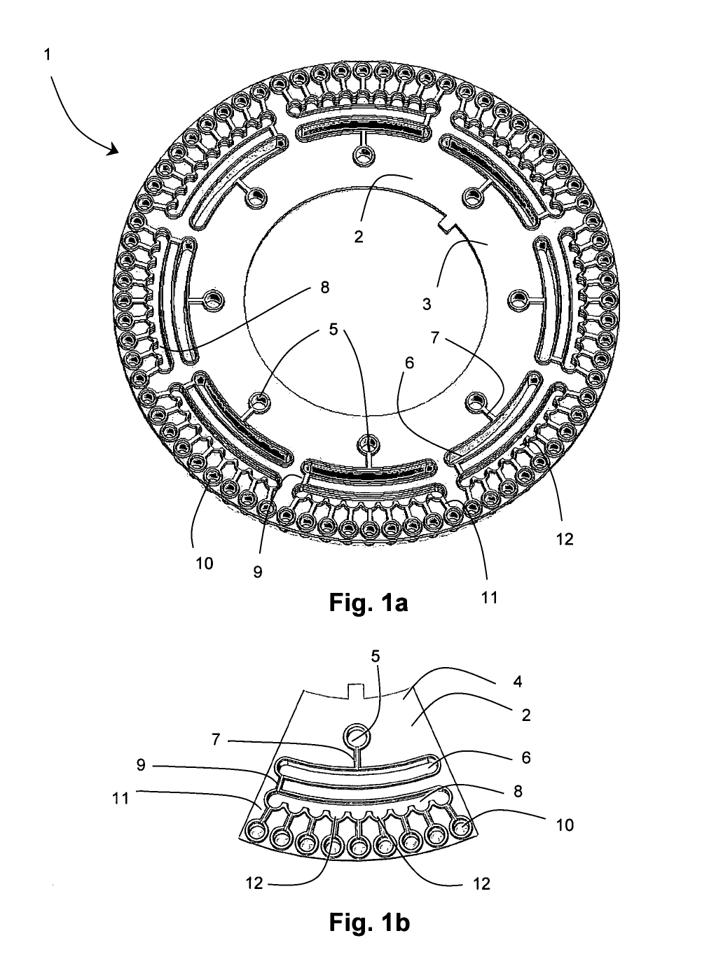

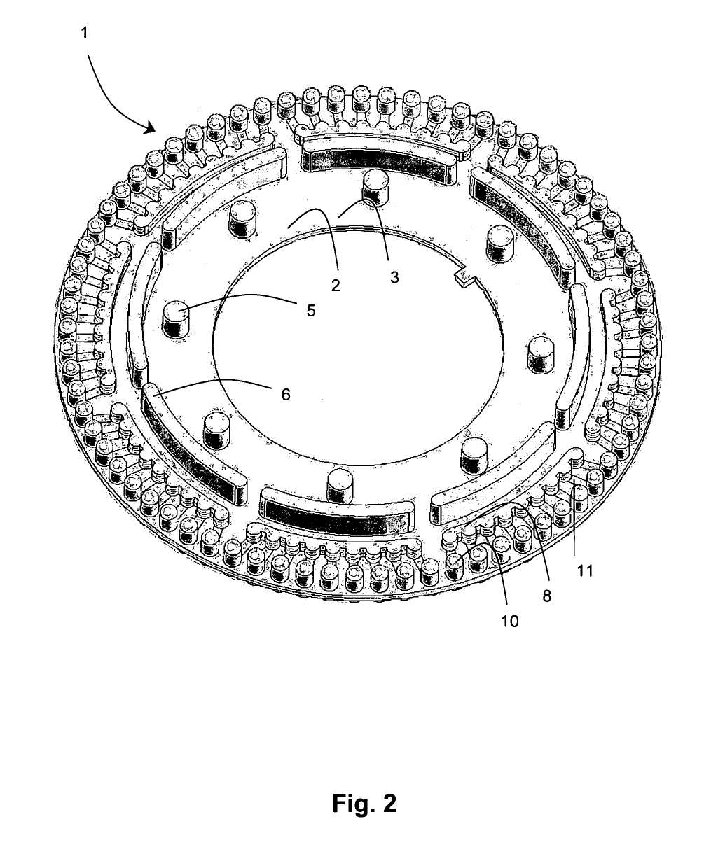

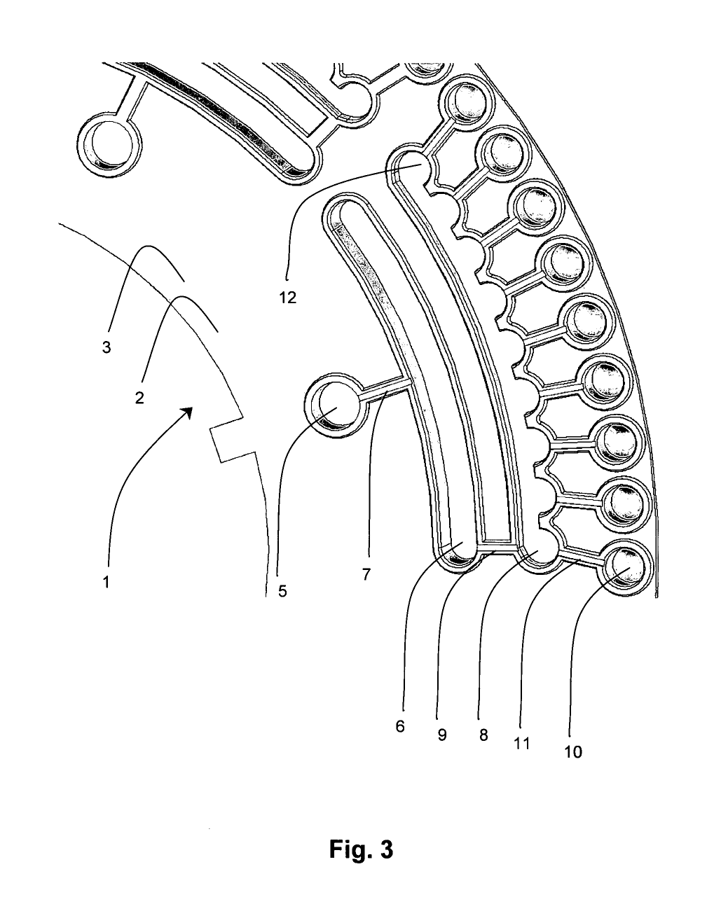

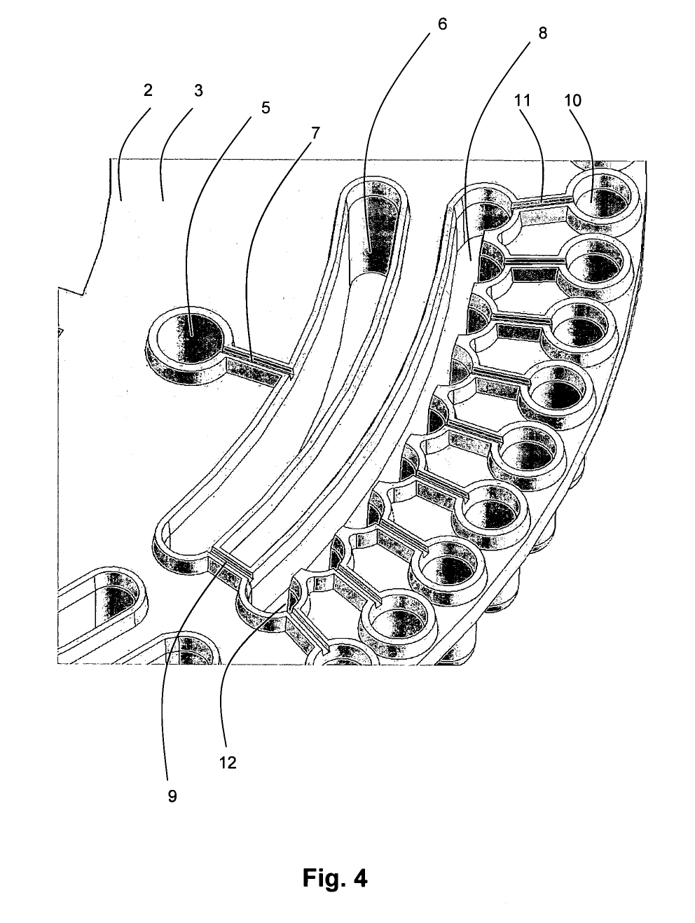

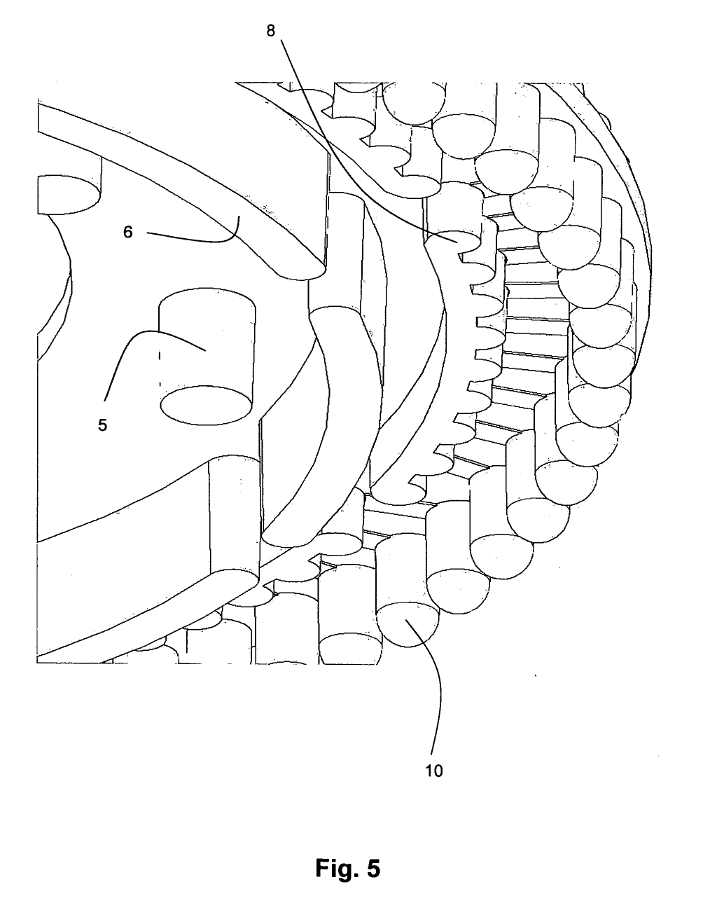

References will now be made to the drawings wherein like reference numerals refer to like parts throughout. Referring to the drawings, the present invention provides apparatus 1 for conducting sequential nucleic acid amplifications, comprising a rotatable platform 2 in the form of a generally planar circular or annular disc 3. In one embodiment, as best shown in FIG. 1b, the rotatable platform 2 is a sector or segment of a circular/annular disc i.e. a wedge 4. In the embodiment shown in FIG. 1a, the rotatable platform 2 includes a plurality of radially inner sample compartments 5 for receiving a plurality of fluid samples/reaction mixtures for conducting first round multiplex amplification reactions. Each of the sample compartments 5 are fluidly connected with a respective dilution compartment 6 by a channel 7, which is in turn in fluidly connected with a respective metering manifold 8 by a channel 9 for substantially evenly distributing each diluted fluid sample into a respective array of radially outer reaction compartments 10 for conducting a plurality of second round nested or hemi-nested amplification reactions. Each of the reaction compartments 10 are fluidly connected to a respective metering manifold 8 by a channel 11 having a selective flow means (not shown) for selectively permitting a fluid flow therethrough.

The substantially radially extending channels 7, 9 and 11 are provided for fluidly connecting the sample compartment 5 to the dilution compartment 6, the dilution compartment 6 to the metering manifold 8, and the metering manifold 8 to the reaction compartments 10, respectively. The channels 7, 9 in series fluidly connect the sample compartment 5, dilution compartment 6 and metering manifold 8, whilst channels 11 in parallel fluidly connect each of the reaction compartments 10 with the metering manifold 8. However it will be appreciated that other configurations for fluidly connecting the compartments will be possible to similar effect.

The sample compartment 5 is preferably disposed at a radially inner position and the plurality of reaction compartments 10 are preferably substantially evenly circumferentially spaced about the periphery of the disc 3; the dilution compartment 6 and metering manifold 8 being disposed therebetween. This arrangement of compartments generally defines a sector of the disc 3. The disc 3 may comprise a plurality of sectors. For example, the disc may include between about 2 to 20 sectors having about 72 reaction compartments 10 in total. However, the disc may include as few as 18 and as many as 144 reaction compartments 10. As the skilled person will appreciate, the number of compartments may be tailored to suit the particular application/assay.

The volumetric capacity of the sample compartment 5 is from about 5 μL to about 20 μL and the dilution compartment 6 from about 100 μL to about 500 μL. The total volume of the metering manifold 8 is preferably about 1200 μL and the combined volume of the axially extending “funnels” 12 disposed on the radially outer wall of the metering manifold 8 totals about 100 μL to about 600 μL. Each of the reaction compartments 10 have a volumetric capacity of about 20 μL to about 250 μL. The sample compartment 5 and the reaction compartments 10 are formed as substantially axially extending cylindrical wells. However, the dilution compartment 6 and the metering manifold 8 are preferably formed as substantially arcuate troughs (when viewed axially).

The initial fluid sample may be successively transported from the sample compartment 5 to the dilution compartment 6, then to the metering manifold 8 and finally to the reaction compartments 10. The driving force for transporting the fluid from compartment to compartment is the application of centrifugal force by rotation of the platform 2. The platform may be rotated at a “low” speed to initially move the fluid sample from the sample compartment 5 to the dilution compartment 6, and then at a “high” speed to move the diluted fluid sample from the dilution compartment 6 to the metering manifold 8. The “low” speed may be about 200 to about 600 rpm and the “high” speed greater than about 1000 rpm. However the skilled person will appreciate that other rotational speeds may be employed for similar effect. Additionally, since during centrifugation under acceleration the fluid sample will tend to migrate to one end of the dilution compartment 6 (depending on the direction or rotation) the channel 7 fluidly connecting the dilution compartment 6 and the metering manifold 8 is preferably disposed at a common end of these compartments, rather than substantially centrally disposed. This configuration advantageously allows a selective flow between these compartments depending on the direction of rotation.

A selective flow means (not shown) is provided for selectively permitting a fluid flow from the metering manifold 8 to the plurality of reaction compartments 10. The selective flow means is a preferably a wax valve having a predetermined melting temperature of about 95° C. Thus, raising the local temperature in the region surrounding the wax valve above the predetermined melting temperature melts the wax and opens the channel thereby to permit a fluid flow therethrough. Advantageously, melting the wax provides a surface seal on the top of the fluid sample since the wax is of typically lower density than the fluid sample.

The present invention also provides a method for conducting nucleic acid amplification, comprising providing apparatus 1 as described above, charging the sample compartment 5 with a fluid sample and charging the dilution compartment 6 with a dilution liquid and then conducting a first round multiplex amplification reaction. Sufficient centrifugal force is then applied to transport the fluid sample to the dilution compartment 6 for diluting the sample. Such as with second round reaction components including buffer, additives and the like as known in the art, but not including second round primers. Additional centrifugal force is then applied to transport the diluted fluid sample to the metering manifold 8. The selective flow means is then actuated and further centrifugal force is applied to distribute the diluted fluid sample to the reaction compartments 10. A plurality of simultaneous second round nested or hemi-nested amplification reactions are then conducted.

It will be appreciated that the annular platform 2 or the wedge-shaped platform 4 as discussed above may be received in a complementary rotatable base (not shown) which provides a centrifugal force for transporting fluid and optionally for heating/cooling the various compartments for conducting the nucleic acid amplifications, for example by RotorGene® equipment marketed by Corbett Research (See International Patent Application No. PCT/AU98/00277). Alternatively, the sample compartment 5, dilution compartment 6, metering manifold 8, reaction compartments 10 and interconnecting channels 7, 9 and 11 are provided separately such that they can be fitted or clipped into receptacles in suitable rotor equipment.

The Rotor-Gene® equipment may comprise a base adapted to receive the apparatus 1, a rotating means for rotating the apparatus 1 according to a pre-determined sequence to selectively transport fluid from one compartment to the next, temperature control elements in thermal communication with the sample 5 and reaction compartments 10 for providing a thermal profile sequence for conducting the first round multiplex and subsequent second round nested or hemi-nested amplification reactions respectively, and a programmable controller for storing and controlling the sequences. However, it will be appreciated that other equipment may be adapted for the present invention.

The rotatable platform 2 is preferably formed from a thermostable thermoplastics material such as polypropylene or polyethylene which is preferably injection moulded. Advantageously, the thermoplastics material may be transparent at predetermined wavelengths for allowing a light source/detector arrangement to monitor a reaction occurring in a compartment 5 or 10.

### EXAMPLES

The present invention will now be described with reference to the following examples which should be considered in all respects as illustrative and non-restrictive.

In one example, multiplexed primer pairs are lyophilsed in the sample compartment 5. Individual second round amplification primer pairs are also lyophilised into each of the reaction compartments 10. The reaction compartments 10 having primer pairs are nested or hemi-nested inside the primer pairs used in the sample compartment 5 to constitute an MT-PCR reaction. After lyohpilisation of primers the apparatus 1 is sealed with a plastic sheet (not shown) leaving apertures positioned above the reaction compartment 10 for addition of components to initiate the reaction, and the dilution compartment 6 to allow introduction of diluent(s).

Nulceic acid sample and mastermix are introduced into the reaction compartments 5 with oil overlay. The second round master mix (MMX2) is introduced into the dilution compartment 6, and the apparatus 1 is placed inside modified Rotor-Gene® equipment. This RotorGene® equipment has a circumferential heater in contact with the base of the reaction compartments 5. Sectors of the circumferential heater can be maintained at different temperatures using electrical resistance heaters and/or peltier devices.

The circumferential heater may also contain a light source and detector to monitor the progress of the reactions using an intercalating fluorescent dye or UV absorption to measure the amount of DNA produced. This can be used to prevent excessive amplification of targets during the multiplexed PCR reaction.

The apparatus 1 is rotated at low speed (e.g. 1 revolution per minute) using a stepper motor. This moves the fluid in the reaction compartments 5 over different temperature zones to achieve PCR amplification of the multiplexed inner primers. After the required number of cycles the apparatus 1 is rotated at medium speed to move the contents of the reaction compartments 5 into the dilution compartment 6. No wax seal is required, as low centrifugal force is required to urge fluid from the sample compartments 5 to the dilution compartment 6. Preferably the reaction compartments 5 have inclined walls to ensure that the sample flows into the dilution compartment 6 with relative low force i.e. 200 to 600 rpm. Alternate clockwise and counter clockwise rotation can be used to thoroughly mix the diluted reaction fluid or the stepper motor may be vibrated.

Rotation of the apparatus 1 at high speed (i.e. greater than 1000 rpm) transports the diluted reaction fluid into the metering manifold 8. Note that the diluted reaction fluid cannot enter the reaction compartments 10 due to the wax interrupting the channels 11 fluidly connecting the metering manifold 8 to the reaction compartments 10. Heating the channels 11 between the dilution compartment 6 and the reaction compartments 10 to 95° C. melts the wax and high speed rotation of the apparatus 1 at this temperature transports the reaction fluid into the reaction compartments 10. Here the diluted master mix comes into contact with the individual primer pairs lyophilised into each of the reaction compartments 10 allowing the lyophilised primers to dissolve into the second round PCR reaction mix and allowing a second nested or hemi-nested PCR reaction to proceed. This reaction can be monitored in real time using intercalating dyes, fluorescent probes or using high resolution melt analysis.

It will be appreciated that the design of the apparatus 1 provides that MMX2 contained in the dilution compartment 6 does not enter the metering manifold 8 at low RPM (200 to 600 rpm). Also, it will be appreciated that the wax forms a physical barrier to ensure reliable dosing and also acts to eliminate evaporation during the second round of PCR. Further, the wax sits on top of the reaction volume in the reaction compartments 10.

In another example, RotorGene® equipment was modified to include an annular heating element such that the element was disposed subjacent the reaction compartments. The annular heating element was formed from aluminium with an embedded electrical heater. To heat, a current was applied to the annular heating element, and to cool an RG cooling system was employed, i.e. a cooling blower was used to pass air through heat sink fins disposed underneath the annular heating element. The apparatus 1 was spun at low rpm, e.g. 1 revolution per minute to simultaneously locally heat the sample compartments 5 and to complete the first round of PCR enrichment.

Once the first round of PCR enrichment was complete, the annular heating element would drop away, so the heater surface moves flush to the bottom of the RG chamber, then the RG functions as normally.

The apparatus 1 was spun with clockwise acceleration to transport the fluid sample to the dilution compartment 6. The apparatus 1 was then agitated and spun again with acceleration (anti-clockwise acceleration) to transport the diluted fluid sample to the metering manifold 8. Once the diluted fluid sample equalizes in each of the dosing funnels the RotorGene® equipment was used to heat (using the regular convection process of air heating and cooling) the apparatus 1 to melt the wax and spun to transport the fluid sample to the reaction compartments 10. Then the RotorGene® equipment was used to conduct a second round of nested or hemi-nested PCR.

Although the invention has been described with reference to specific examples, it will be appreciated by those skilled in the art that the invention may be embodied in many other forms.

1. Apparatus for conducting sequential nucleic acid amplification reactions, the apparatus comprising:
a platform comprising:
a sample compartment adapted to receive a fluid sample for conducting a first amplification reaction, and
a plurality of reaction compartments,
said platform being adapted to substantially evenly distribute said fluid sample into said plurality of reaction compartments for conducting second amplification reactions.

2. Apparatus according to claim 1 wherein said platform is rotatable for generating a centrifugal force.

3. Apparatus according to claim 1 wherein said sample compartment is positioned radially inwards with respect to said reaction compartments.

4. Apparatus according to claim 1 further including a dilution compartment fluidly connected to said sample compartment and said reaction compartments.

5. Apparatus according to claim 1 including a metering manifold and a dilution compartment, wherein said sample compartment is fluidly connected to said dilution compartment, and said dilution compartment is fluidly connected to said metering manifold, and said metering manifold is fluidly connected to said plurality of reaction compartments.

6. Apparatus according to claim 5 including substantially radially extending channels for providing said fluid connections between said sample compartment and said dilution compartment and said metering manifold.

7. Apparatus according to claim 6 wherein said sample compartment, dilution compartment and metering manifold are fluidly connected in series and each said reaction compartment is fluidly connected with said metering manifold in parallel.

8. Apparatus according to claim 6 wherein said platform is a circular or annular disc and said sample compartment, dilution compartment, metering manifold and reaction compartments are embedded in the surface of said circular or annular disc.

9. Apparatus according to claim 8 wherein said plurality of reaction compartments are circumferentially spaced about the periphery of said disc.

10-14. (canceled)

15. Apparatus according to claim 6 wherein said fluid sample flows through said channels when said disc is rotated at a sufficient rpm.

16. Apparatus according to claim 15 wherein said number of rpm is between about 200 to 1000 rpm.

17. Apparatus according to claim 6 wherein said channel fluidly connecting each said reaction compartment to said metering manifold includes a selective flow means for selectively permitting a fluid flow therethrough.

18-20. (canceled)

21. Apparatus according to claim 6 wherein said sample compartment and said reaction compartment are formed as substantially cylindrically shaped wells, and said dilution compartment and said metering manifold are formed as substantially arcuate troughs.

22. Apparatus according to claim 21 wherein said channel fluidly connecting said dilution compartment to said metering manifold is disposed at a common end of said arcuate troughs for allowing selective movement of fluid from said dilution compartment to said metering manifold depending upon the direction of rotation of said apparatus under acceleration.

23-29. (canceled)

30. Apparatus according to claim 6 wherein said wells comprise an inclined wall portion such that the fluid tends to drain to said channel leading to a subsequent compartment/manifold when centrifugal force is applied by rotation of said disc.

31. Apparatus according to claim 6 wherein said channels have progressively smaller diameters from the inner to the outer compartment such that a greater number of rpm are required to transport said fluid from one compartment/manifold to the next.

32. Apparatus according to claim 1 wherein said sample and reaction compartments are adapted for thermal communication with a heat exchanger for conducting said first and second amplification reactions respectively.

33. Apparatus according to claim 1 wherein said first amplification reaction comprises a multiplex PCR reaction and said second amplification reaction comprises a nested or hemi-nested PCR reaction.

34. Apparatus for conducting sequential nucleic acid amplification reactions, the apparatus comprising: a rotatable platform having a plurality of sample compartments adapted to adapted to receive a fluid sample for conducting a first amplification reaction, each said reaction compartment being fluidly connected with a respective dilution compartment, each respective dilution compartment in turn being fluidly connected with a respective metering manifold for substantially evenly distributing each said diluted fluid sample into a respective array of reaction compartments for conducting a plurality of second amplification reactions, each said reaction compartment being fluidly connected to a respective metering manifold by a channel having a selective flow means for selectively permitting a fluid flow therethrough.

35. Apparatus according to claim 34 wherein said plurality of sample compartments are positioned radially inwards of said reaction compartments, and wherein the application of sufficient centrifugal force selectively transports said fluid sequentially from compartment to compartment, or from compartment to manifold, or from manifold to compartment.

36-37. (canceled)

38. A device for conducting nucleic acid amplification, comprising: a base for receiving said apparatus according to claim 1; a rotating means for rotating said apparatus according to a pre-determined sequence to selectively transport said fluid sample from one compartment/manifold to the next; temperature control elements in thermal communication with said fluid sample and said reaction and sample compartments for providing a thermal profile sequence for conducting said first and second amplification reactions; and a programmable controller for storing and controlling said sequences.

39-41. (canceled)
